(19) United States
(12) Patent Application Publication
(10) Pub. No.: US 2025/0041484 A1
(43) Pub. Date: Feb. 6, 2025

(54) PREPARATION METHOD FOR BIOLOGICAL MEMBRANE, AND PRODUCT AND APPLICATION THEREOF

(71) Applicant: DB WUDEREGEN BIOMEDICAL TECHNOLOGIES (JIANG SU) CO., LTD, Jiangsu (CN)

(72) Inventors: Qun DONG, Jiangsu (CN); Hengfei REN, Jiangsu (CN)

(21) Appl. No.: 18/729,951

(22) PCT Filed: May 23, 2022
(86) PCT No.: PCT/CN2022/094367

§ 371 Date: Jul. 18, 2024

(30) Foreign Application Priority Data
Jun. 10, 2021 (CN) 202110647054.7

(51) Int. Cl. A61L 27/36

(52) U.S. Cl. CPC A61L 27/3687; A61L 27/3604; A61L 27/3629; A61L 27/3691; A61L 2430/34; A61L 2430/40

(57) ABSTRACT

The present application provides a preparation method for a biological membrane, and a product and an application thereof. The preparation method for the biological membrane comprises the following steps: (1) treating animal cavity tissues by using a collagenase inhibitor; (2) removing a membrane layer which accounts for more than 40% of the total fat content of the animal cavity tissues; (3) using a first enzyme solution to treat a product obtained in step (2); (4) using an alkali solution to treat a product obtained in step (3); (5) using a decellularized solution to treat a product obtained in step (4); (6) using a degreasing fluid solution to treat a product obtained in step (5); (7) using a second enzyme solution to treat a product obtained in step (6); and (8) using supercritical carbon dioxide to clean a product obtained in step (7) to obtain a biological membrane. In the present application, the integrity of a multi-layer space structure, elastic fibers and a collagen network of the biological membrane can be reserved to a great extent, the mechanical strength is high, residues of organic matters and reagents in the product can be removed, and the tissue regeneration inducing effect of the product is better.

### TECHNICAL FIELD

The present application relates to the field of biotechnology, specifically to a preparation method for a biological membrane and a product therefrom and an application thereof, and in particular, relates to a preparation of a multilayered natural biological membrane based on animal lumen-containing organs tissue and its application in guiding tissue regeneration.

### BACKGROUND

Advances and developments in tissue engineering technology light the hope for repairing vital tissues and organs such as urinary system reconstruction, digestive system reconstruction, and female genital tract reconstruction. Tissue engineering technology encompasses the fields of scaffold materials, seed cells, and growth factors. The extracellular matrix is a good scaffold material. The extracellular matrix contains collagen network structure and elastic fibers, etc., which are conducive to cell adhesion and growth, and have promising prospects in clinical application. In recent years, many studies have used biological enzymes and reagents to remove cells and cellular debris from bladders or dermal tissues and retain the extracellular matrix which forms a cell-free matrix structure abundantly having collagen, elastin, elastic fibers, and vascular framework. Such material has good biocompatibility, mechanical stability, and also low immunogenicity, thereby providing a good scaffold for cell regeneration and creating an excellent microenvironment required for cell regeneration.

CN107397978A discloses a preparation method for a decellularized matrix of animal lumen-containing organs tissue, which is prepared by subjecting the submucosa of an animal lumen-containing organs tissue to viral inactivation step, defatting step, and decellularization step. In the method, the step of decellularization includes several times of freezing and thawing steps and decellularization steps with a decellularization solution carried out after several times of freezing and thawing steps. The decellularization and defatting treatments of this method suffer from the problems of large dosages of chemicals, incomplete decellularization, high residues and high toxicity of chemicals, and low mechanical properties of the prepared material.

CN106256382A discloses a preparation method for a bladder biological scaffold, comprising (1) treating the bladder tissue with NaOH; (2) treating the resulting material with TritonX-100 and SDS in any order and repeating this step for once; (3) treating the resulting material with NaOH; and (4) drying the resulting material at low temperature. The decellularization and defatting treatments of this method also suffer from the problems of large dosages of chemicals, incomplete decellularization, high residues and high toxicity of chemicals, and low mechanical properties of the prepared material; additionally, the preparation period is extremely long due to the retention of partial muscularis and affected by the fat (the process needs 45 days to complete the preparation).

CN107164298A discloses a preparation method for a soft tissue decellularized matrix, relating to the technical field of clinical medicine, biomedicine, and regenerative medicine. The preparation method for a soft tissue decellularized matrix provided by this invention employs supercritical fluid technology to prepare a natural soft tissue decellularized matrix. The preparation method for a soft tissue decellularized matrix provided by this invention has small damage to soft tissue matrix components, and the obtained soft tissue decellularized matrix has low immunogenicity and good biocompatibility.

Therefore, it is essential in this filed to develop a preparation method for a biological membrane, which maximally retains the integrity of the multilayered spatial structure, elastic fibers, and collagen network structure of the biological membrane.

### SUMMARY

The present application provides a preparation method for a biological membrane, and a product therefrom and an application thereof, and in particular provides a preparation method for a biological membrane having a multilayered structure, and a product therefrom and an application thereof. The preparation method described in the present application can efficiently retain the integrity of the multilayered spatial structure, elastic fibers, and collagen network structure of the biological membrane, bring high mechanical strength, remove the residues of organic substances and reagents from the product, and endow the product with better efficiency to guide tissue regeneration; and the overall process to prepare the biological membrane having a multilayered structure needs an extremely short period, only 7 days or less.

In a first aspect, the present application provides a preparation method for a biological membrane; and the preparation method for a biological membrane includes the following steps:
(1) treating an animal lumen-containing organs tissue with a collagenase inhibitor;
(2) removing a membrane layer which contains fat accounting for more than or equal to 40% of the total fat content of the animal lumen-containing organs tissue;
(3) treating a product obtained from step (2) with a first enzyme solution;
(4) treating a product obtained from step (3) with an alkaline solution;
(5) treating a product obtained from step (4) with a decellularization solution;
(6) treating a product obtained from step (5) with a defatting liquid solution;
(7) treating a product obtained from step (6) with a second enzyme solution; and
(8) cleaning a product obtained from step (7) with supercritical carbon dioxide to obtain the biological membrane.

In the present application, the collagenase inhibitor treatment is employed to protect the collagen fibers of the lumen-containing organs tissue, and then a physical method (mechanical excision) is employed to remove a high proportion of fat in the lumen-containing organs tissue structure, and this step can remove fat of 40% or more of the total fat content of the animal lumen-containing organs tissue (preferably 40-60%, said content is a content by mass percentage). In addition, most animal lumen-containing organs tissues have a dense serosa structure, and the physical removal of the dense serosa can improve the efficiency of decellularization and further chemical defatting and greatly save the time of biological membrane preparation. In the present application, mild and low-toxic reagents are used sequentially to carry out the steps of decellularization and defatting, decreasing the damage of reagents to the biological membrane; the enzyme method is used to remove impurity proteins, nucleic acids, polysaccharides, and other substances that can cause immune reactions; supercritical CO₂ fluid extraction technology is introduced to remove the residues of organic substances and reagents from the product; on one hand, the method can efficiently retain the integrity of the multilayered spatial structure, elastic fibers, and collagen network structure of the biological membrane, bringing high mechanical strength, and on the other hand, the non-toxic and environmental-friendly supercritical CO₂ fluid extraction technology is introduced to remove the residues of organic substances and reagents from the product, endowing the product with better efficiency to guide tissue regeneration.

In the method, the collagenase inhibitor is used for the treatment in step (1). Collagenase relies on some metal ions such as calcium, zinc, and magnesium to maintain its normal structure and function, and when these ions are complexed by the collagenase inhibitor, the activity of collagenase can be inhibited to protect collagen protein from damage and thusly to protect the collagen fibers of the animal lumen-containing organs tissue, avoiding the influence of mechanical heating and chemical reagents on the structure of collagen fibers during subsequent mechanical and chemical processes. It is found that most of the fat in the animal lumen-containing organs tissue is distributed in the serosa and the subserosa, so the mechanical excision used to remove the serosa and subserosa of the animal lumen-containing organs in step (2) is able to remove 40% or more of the fat (preferably 40-60%, said content is a content by mass percentage), which lays the groundwork for further chemical defatting; additionally, the removal of the dense serosa facilitates the enzyme solution, decellularization reagent, and detergent entering into the inner wall of the tissue, which greatly improves the removal efficiency of muscle cells, impurity proteins, and other immunogenic components, and thereby the time of biological membrane preparation is significantly reduced (reduced from 50 days in the prior art to less than or equal to 7 days directly).

In the method, steps (3)-(7) are in the order of using a first enzyme solution, an alkaline solution, a decellularization solution, a defatting liquid solution, and a second enzyme solution for the treatment. Because the use of the first enzyme solution can play a role in digesting the connections between muscle cells, which facilitates muscle cells lysis; moreover, the collagen fibers on the tissue wall of animal lumen-containing organs are loosened by the alkaline solution, and thus the decellularization solution can effectively enter the tissue wall and combine with the phospholipids on the cell membrane to rupture the cells, so as to completely remove the muscle cells and cellular debris. After the cells are removed, the pore size of the material becomes larger, and thus the defatting liquid can effectively enter the tissue pores and effectively remove the fat remaining on the inner tissue wall. Finally, the use of the second enzyme solution can remove the remnant impurity proteins, polysaccharides, and nucleic acids from the scaffold material, which is conducive to reducing the immunogenicity of the material. The above method is able to minimize the amount of chemicals, and further avoids the problems of high toxicity or high residue level of the chemicals used as well as the low mechanical properties of the prepared material, and maximally retains the integrity of the multilayered spatial structure, elastic fibers, and collagen network structure of the biological membrane.

In the method, supercritical carbon dioxide cleaning is employed in step (8). The supercritical fluid extraction technology (SFE) is a fast-developing, widely used new separation technology, with advantages of simple operation, low energy consumption, no pollution, good separation efficiency, low cost, and no solvent residue. In view of these advantages of the supercritical CO₂ fluid extraction technology, the present application employs the supercritical CO₂ fluid extraction technology for cleaning in the process of preparing the multilayered natural biological membrane of animal lumen-containing organs tissue, in order to remove the residues of the organic solvents and reagents, and at the same time, to further remove some substances with immunogenicity such as polysaccharides, accordingly increasing the guiding activity of the multilayered natural biological membrane in guiding the tissue regeneration.

Preferably, the animal lumen-containing organs tissue in step (1) includes serosa, subserosa, muscularis, submucosa, and mucosa sequentially layered.

Preferably, the animal lumen-containing organs tissue in step (1) is selected from any one of an animal esophageal tissue, an animal stomach tissue, an animal intestine tissue, an animal urethral tissue, or an animal bladder tissue.

In the present application, the animal lumen-containing organs tissue may be selected from, but is not limited to, any one of porcine lumen-containing organs tissue, bovine lumen-containing organs tissue and ovine lumen-containing organs tissue.

Preferably, the collagenase inhibitor in step (1) includes any one or a combination of at least two of acetylcysteine, disodium edetate, penicillamine, medroxyprogesterone acetate, sodium citrate, tetracycline, or doxycycline.

Preferably, the treatment in step (1) specifically is: treating the animal lumen-containing organs tissue by immersing it in an aqueous solution of the collagenase inhibitor.

Preferably, the aqueous solution of the collagenase inhibitor has a mass concentration of 1-20 wt %, such as 1 wt %, 2 wt %, 5 wt %, 10 wt %, 15 wt %, 20 wt %, etc.

Preferably, the animal lumen-containing organs tissue and the aqueous solution of the collagenase inhibitor have a mass ratio of 1:(5-15), such as 1:5, 1:6, 1:7, 1:8, 1:9, 1:10, 1:11, 1:12, 1:13, 1:14, 1:15, etc.

Preferably, the immersion is carried out at a temperature of 20-30° C., such as 20° C., 22° C., 24° C., 25° C., 26° C., 28° C., 30° C., etc., and the immersion is carried out for a period of 1-6 h, such as 1 h, 2 h, 3 h, 4 h, 5 h, 6 h, etc.

Preferably, the removal in step (2) is carried out in a manner of any one of mechanical cutting device excision, scissors trimming, manual peeling, or physical grinding, preferably mechanical cutting device excision.

For the removal, the advantages of choosing mechanical cutting device excision are: the removal or retaining of membrane layers can be precisely controlled as wanted; the goal of physical defatting can be effectively achieved; the time of preparing the biological membrane can be further shortened; and compared with other existing removal methods, the mechanical cutting device excision can more precisely control the thickness of the processed membrane layer and the surface homogeneity of the membrane layer.

Preferably, specific process parameters for the mechanical cutting device excision include: a voltage of 160-265 V (such as 160 V, 180 V, 200 V, 220 V, 240 V, 260 V, 265 V, etc.), a power of 1200-4000 W (such as 1200 W, 2000 W, 2500 W, 3000 W, 3500 W, 4000 W, etc.), a cutting rate of 5-40 mm/s (such as 5 mm/s, 10 mm/s, 15 mm/s, 20 mm/s, 25 mm/s, 30 mm/s, 35 mm/s, 40 mm/s, etc.), and a roughness of tissue surface after excision of less than 100 μm (such as 99 μm, 95 μm, 90 μm, 98 μm, 80 μm, 75 μm, 70 μm, 65 μm, 60 μm, 55 μm, 50 μm, 40 μm, 30 μm, 20 μm, 10 μm, etc.).

For the removal, the mechanical device cutting carried out with such parameters gives the biological membrane lower roughness and more uniform surface.

Preferably, the removal in step (2) is removing the membrane layer which contains fat accounting for 40-60% of the total fat content of the animal lumen-containing organs tissue (such as 40%, 41%, 42%, 43%, 44%, 45%, 46%, 47%, 48%, 49%, 50%, 51%, 52%, 53%, 54%, 55%, 56%, 57%, 58%, 59%, 60%, etc.), preferably 44-52%.

Preferably, the membrane layer removed in step (2) includes serosa and subserosa.

Preferably, the membrane layer removed in step (2) further includes a part of muscularis.

Preferably, the membrane layer removed in step (2) is serosa and subserosa, and the product obtained is muscularis, submucosa and mucosa; or the membrane layer removed is serosa, subserosa, and a part of muscularis, and the product obtained is a remaining part of muscularis, submucosa, and mucosa.

The “part of muscularis” refers to muscularis with a fat content of 10-20% (such as 10%, 12%, 14%, 16%, 18%, 20%, etc.) relative to the total fat content, and a thickness of approximately 0.5-1.5 mm (such as 0.5 mm, 0.6 mm, 0.7 mm, 0.8 mm, 0.9 mm, 1.0 mm, 1.1 mm, 1.2 mm, 1.3 mm, 1.4 mm, 1.5 mm, etc.), and selectively removing a part of muscularis can result in biological membranes of different thicknesses for different indications.

Preferably, the membrane layer removed in step (2) has a total thickness of 1-3 mm, such as 1 mm, 1.2 mm, 1.4 mm, 1.6 mm, 1.8 mm, 2 mm, 2.2 mm, 2.4 mm, 2.6 mm, 2.8 mm, 3 mm, etc.

In the present application, the mechanical defatting only removes the serosa and the subserosa of the animal lumen-containing organs tissue but retains a part of the muscularis, the purpose of which is: the extracellular matrix components of the muscularis is able to better simulate the microenvironment of the extracellular matrix, and provides a good scaffolding for the cell regeneration; moreover, the muscularis and connective tissues account for a large proportion of the whole bladder wall, and after removing the muscle cells in the muscularis, the pore size of muscle extracellular matrix becomes larger, and the collagen fibers and elastin are loose in arrangement, which is conducive to better growth of various tissue cells, and contributes to a wide application in the repair and regeneration of different tissues.

Preferably, the first enzyme solution in step (3) includes a trypsin solution and/or a protease 1398 solution.

Preferably, the first enzyme solution in step (3) has a concentration of 0.1-5 wt %, such as 0.1 wt %, 0.5 wt %, 1 wt %, 1.5 wt %, 2 wt %, 2.5 wt %, 3 wt %, 3.5 wt %, 4 wt %, 4.5 wt %, 5 wt %, etc.

Preferably, the treatment in step (3) is carried out in a manner of immersion, and the immersion is carried out at a temperature of 20-30° C., such as 20° C., 22° C., 24° C., 26° C., 28° C., 30° C., etc., and the immersion is carried out for a period of 1-8 h, such as 1 h, 2 h, 3 h, 4 h, 5 h, 6 h, 7 h, 8 h, etc.

Preferably, the alkaline solution in step (4) includes any one or a combination of at least two of a sodium hydroxide solution, a potassium hydroxide solution, a sodium carbonate solution, or a calcium hydroxide solution.

Preferably, the alkaline solution in step (4) has a concentration of 0.1-5 mol/L, such as 0.1 mol/L, 0.2 mol/L, 0.5 mol/L, 1 mol/L, 1.2 mol/L, 1.5 mol/L, 2 mol/L, 2.5 mol/L, 3 mol/L, 3.5 mol/L, 4 mol/L, 4.5 mol/L, 5 mol/L, etc.

Preferably, the alkaline solution in step (4) further includes a collagenase inhibitor of 1-5 wt % (such as 1 wt %, 1.5 wt %, 2 wt %, 2.5 wt %, 3 wt %, 3.5 wt %, 4 wt %, 4.5 wt %, 5 wt %, etc.).

Preferably, the collagenase inhibitor includes any one or a combination of at least two of acetylcysteine, disodium edetate, penicillamine, medroxyprogesterone acetate, sodium citrate, tetracycline, or doxycycline.

Preferably, the treatment in step (4) is carried out in a manner of immersion, and the immersion is carried out at a temperature of 4-25° C., such as 4° C., 5° C., 6° C., 7° C., 8° C., 9° C., 10° C., 11° C., 12° C., 13° C., 14° C., 15° C., 17° C., 19° C., 21° C., 23° C., 25° C., etc., and the immersion is carried out for a period of 1-4 h, such as 1 h, 1.5 h, 2 h, 2.5 h, 3 h, 3.5 h, 4 h, etc.

Preferably, the decellularization solution in step (5) includes an aqueous solution of a surfactant.

Preferably, the surfactant includes any one or a combination of at least two of polysorbate (also known as Tween), sodium dodecylaminopropionate, polyethylene glycol octylphenyl ether (TritonX-100), or alkylphenol polyoxyethylene ether.

Preferably, the polysorbate (also known as Tween) includes any one or a combination of at least two of polysorbate-20 (Tween-20), polysorbate-40 (Tween-40), polysorbate-60 (Tween-60), or polysorbate-80 (Tween-80).

Preferably, the aqueous solution of the surfactant has a concentration of 0.1-5 wt %, such as 0.1 wt %, 0.5 wt %, 1 wt %, 1.5 wt %, 2 wt %, 2.5 wt %, 3 wt %, 3.5 wt %, 4 wt %, 4.5 wt %, 5 wt %, etc.

Preferably, the decellularization solution further includes a collagenase inhibitor of 1-5 wt % (such as 1 wt %, 1.5 wt %, 2 wt %, 2.5 wt %, 3 wt %, 3.5 wt %, 4 wt %, 4.5 wt %, 5 wt %, etc.).

Preferably, the collagenase inhibitor includes any one or a combination of at least two of acetylcysteine, disodium edetate, penicillamine, medroxyprogesterone acetate, sodium citrate, tetracycline, or doxycycline.

Preferably, the treatment in step (5) is carried out in a manner of immersion, and the immersion is carried out at a temperature of 15-25° C., such as 15° C., 16° C., 18° C., 20° C., 22° C., 24° C., 25° C., etc., and the immersion is carried out for a period of 5-24 h, such as 5 h, 6 h, 8 h, 10 h, 12 h, 14 h, 16 h, 18 h, 20 h, 22 h, 24 h, etc.

Preferably, the defatting liquid solution in step (6) includes an organic solvent and/or an aqueous solution of a detergent.

Preferably, the organic solvent includes any one or a combination of at least two of sucrose ester, fatty alcohol polyoxyethylene ether, alkylphenol polyoxyethylene ether, ethanol, ethylene glycol, ethyl acetate, isopropanol, trichloromethane, or acetone, preferably a mixture of ethylene glycol and isopropanol.

Preferably, the aqueous solution of the detergent has a concentration of 0.1-5 wt %, such as 0.1 wt %, 0.5 wt %, 1 wt %, 1.5 wt %, 2 wt %, 2.5 wt %, 3 wt %, 3.5 wt %, 4 wt %, 4.5 wt %, 5 wt %, etc.

Preferably, the defatting liquid solution in step (6) further includes a collagenase inhibitor of 1-5 wt % (such as 1 wt %, 1.5 wt %, 2 wt %, 2.5 wt %, 3 wt %, 3.5 wt %, 4 wt %, 4.5 wt %, 5 wt %, etc.).

Preferably, the collagenase inhibitor includes any one or a combination of at least two of acetylcysteine, disodium edetate, penicillamine, medroxyprogesterone acetate, sodium citrate, tetracycline, or doxycycline.

Preferably, the treatment in step (6) is carried out in a manner of immersion, and the immersion is carried out at a temperature of 10-25° C., such as 10° C., 12° C., 14° C., 16° C., 18° C., 20° C., 22° C., 24° C., 25° C., etc., and the immersion is carried out for a period of 1-24 h, such as 1 h, 2 h, 4 h, 6 h, 8 h, 10 h, 12 h, 14 h, 16 h, 18 h, 20 h, 22 h, 24 h, etc.

Preferably, the second enzyme solution in step (7) includes a mixed aqueous solution of a solution of polysaccharidase, a solution of nuclease, and a solution of protease.

Preferably, the polysaccharidase includes any one or a combination of at least two of non-starch polysaccharidase, laminarinase, or aggrecanase.

Preferably, the nuclease includes a DNA enzyme and/or an RNA enzyme.

Preferably, the protease includes any one or a combination of at least two of papain, neutral protease, protease 1398, or cathepsin.

Preferably, the second enzyme solution in step (7) has a concentration of 0.1-10 wt %, such as 0.1 w %, 0.5 w %, 1 w %, 1.5 w %, 2 w %, 2.5 w %, 3 w %, 3.5 w %, 4 w %, 4.5 w %, 5 w %, 6 w %, 7 w %, 8 w %, 9 w %, 10 w %, etc.

Preferably, the treatment in step (7) is carried out in a manner of immersion, and the immersion is carried out at a temperature of 20-30° C., such as 20° C., 22° C., 24° C., 26° C., 28° C., 30° C., etc., and the immersion is carried out for a period of 1-24 h, such as 1 h, 2 h, 4 h, 6 h, 8 h, 10 h, 12 h, 14 h, 16 h, 18 h, 20 h, 22 h, 24 h, etc.

Preferably, the cleaning with supercritical carbon dioxide in step (8) is carried out at a pressure of 10-50 MPa, such as 10 MPa, 15 MPa, 20 MPa, 25 MPa, 30 MPa, 35 MPa, 40 MPa, 45 MPa, 50 MPa, etc.

Preferably, the cleaning with supercritical carbon dioxide in step (8) is carried out at a temperature of 31-50° C., such as 31° C., 32° C., 34° C., 35° C., 36° C., 38° C., 40° C., 42° C., 44° C., 46° C., 48° C., 50° C., etc.

Preferably, the cleaning with supercritical carbon dioxide in step (8) is carried out at a CO₂ flow rate of 1-10 L/min, such as 1 L/min, 2 L/min, 3 L/min, 4 L/min, 5 L/min, 6 L/min, 7 L/min, 8 L/min, 9 L/min, 10 L/min, etc.

Preferably, step (9) is further carried out after step (8): drying and sterilizing the biological membrane obtained from step (8).

Preferably, the drying is freeze-drying; pre-freezing is carried out at a temperature of −80° C. to −20° C. before the freeze-drying, such as −80° C., −75° C., −70° C., −65° C., −60° C., −55° C., −50° C., −45° C., −40° C., −30° C., −20° C., etc., and the freeze-drying is carried out at a temperature of −50-0° C., such as −50° C., −40° C., −30° C., −20° C., −10° C., 0° C., etc., the freeze-drying is carried out for a period of 24-72 h, such as 24 h, 30 h, 36 h, 40 h, 48 h, 50 h, 60 h, 70 h, 72 h, etc., and the freeze-drying is carried out at a vacuum degree of 1-10 Pa, such as 1 Pa, 2 Pa, 3 Pa, 4 Pa, 5 Pa, 6 Pa, 7 Pa, 8 Pa, 9 Pa, 10 Pa, etc.

Preferably, the sterilization is irradiation sterilization.

Preferably, the irradiation sterilization is specifically Cobalt-60 irradiation sterilization.

Preferably, the Cobalt-60 irradiation sterilization is carried out at an irradiation dose of 15-30 KGy, such as 15 KGy, 16 KGy, 18 KGy, 20 KGy, 22 KGy, 24 KGy, 26 KGy, 28 KGy, 30 KGy, etc.

In a second aspect, the present application provides a multilayered natural biological membrane, and the multilayered natural biological membrane is prepared by the preparation method as described in the first aspect.

In a third aspect, the present application provides an application of the multilayered natural biological membrane as described in the second aspect in the preparation of a material for guiding tissue regeneration.

Compared with the prior art, the present application has the beneficial effects below.
(1) The natural biological membrane material prepared by the method provided in the present application retains the structural framework of extracellular matrix of the muscularis in the lumen-containing organs tissue, and additionally multilayered biological membranes of different thicknesses can be prepared by precisely controlling the excision thicknesses of the serosa and subserosa or the muscularis.
(2) In the present application, the biological membranes, which selectively remove different thicknesses of muscularis, have different matrix frameworks and thereby are competently used for multiple indications and guide regeneration of multiple tissues.
(3) The biological membrane made by the method provided in the present application has apparent dense and loose layers, and the loose layer can better guide the growth of tissue cells and play a role in repair and regeneration.
(4) For the method provided in the present application, firstly a part of the fat is physically removed, bringing an obvious defatting result, thus significantly shortening the time of preparing the biological membrane containing the muscularis, and improving the preparation efficiency.

### BRIEF DESCRIPTION OF DRAWINGS

FIG. 1 is a structural schematic diagram showing the cross-section of an animal lumen-containing organs tissue;

in FIG. 1: 1-serosa, 2-subserosa, 3-muscularis, 4-submucosa, and 5-mucosa.

FIG. 2 is a histogram showing fat contents of each layer in longitudinal sectional view of an animal bladder wall tissue provided by the present application;

in FIG. 2: 1-fat content of the serosa and the subserosa, 2-6-fat contents of the muscularis, and 7-fat content of the mucosa and the submucosa; layers 1-7 each have a thickness of 1 mm.

FIG. 3A is an electron microscope image showing the dense side of the biological membrane prepared in Example 1.

FIG. 3B is an electron microscope image showing the rough side of the biological membrane prepared in Example 1.

FIG. 4A is a histological staining diagram showing the cross-section of the biological membrane prepared in Example 1.

FIG. 4B is a partial enlarged view of the box in FIG. 4A;

in FIG. 4B: 1-extracellular matrix of the mucosa and the submucosa, and 2-extracellular matrix of the muscularis.

FIG. 5A is an electron microscope image showing the dense side of the biological membrane prepared in Example 2.

FIG. 5B is an electron microscope image showing the rough side of the biological membrane prepared in Example 2.

FIG. 6A is an electron microscope image showing the dense side of the biological membrane prepared in Example 3.

FIG. 6B is an electron microscope image showing the rough side of the biological membrane prepared in Example 3.

### DETAILED DESCRIPTION

The technical solutions of the present application are further described below through the accompanying drawings and specific embodiments. It should be clear to those skilled in the art that the specific embodiments are merely used for a better understanding of the present application and should not be regarded as a specific limitation to the present application.

FIG. 1 is a structural schematic diagram showing the cross-section of an animal lumen-containing organs tissue; as shown in FIG. 1, the animal lumen-containing organs tissue can be divided into serosa, muscularis and mucosa. The surfaces of the serosa and mucosa are very dense, and the subserosa, muscularis, and submucosa are distributed with blood vessels, connective tissue, and fat.

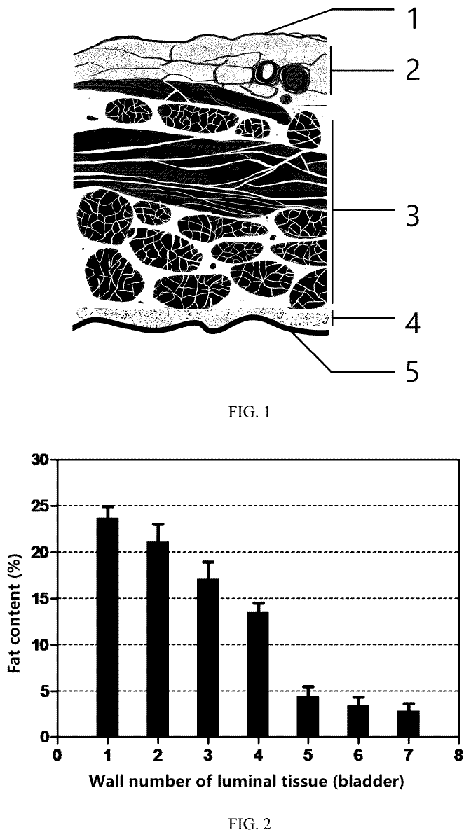

FIG. 2 is a histogram showing fat contents of each layer in a longitudinal sectional view of an animal lumen-containing organs tissue (bladder wall) provided by the present application. As shown in FIG. 2, the fat content of each layer in the lumen-containing organs tissue decreases from the serosa to the mucosa gradually, i.e., most of the fat is contained in the serosa and subserosa of the tissue as well as the muscularis close to the serosa. Therefore, by removing the serosa, the subserosa, and a part of the muscularis, the fat accounting for 40%-60% of the total fat content of the tissue can be removed, which lays the groundwork for further chemical defatting; additionally, the removal of the serosa greatly improves the efficiency of the muscle cell removal.

### Example 1

This example provides a biological membrane; a preparation method for the biological membrane includes the following steps:
(1) 100 g of fresh porcine bladder tissue was immersed in 1 L of a 10 wt % acetylcysteine solution for 2 h;
(2) the product obtained from step (1) was processed at 25° C. by a slicing device to excise a membrane layer of 1 mm thickness from the serosa side of the bladder material, wherein specific process parameters of the mechanical cutting device excision were: a voltage of 220 V, a power of 3600 W, a cutting rate of 15 mm/s, and a roughness of tissue surface after excision of less than 100 μm;
(3) 100 g of the product obtained from step (2) was immersed in 1 L of a 2 wt % trypsin solution at 25° C. for 4 h;
(4) the product obtained from step (3) was immersed in 1 L of a mixed aqueous solution containing 1 mol/L sodium hydroxide and 3% acetylcysteine by mass at 5° C. for 2 h;
(5) the product obtained from step (4) was immersed in 1 L of a decellularization solution at 25° C. for 16 h, wherein the decellularization solution was a mixed aqueous solution of 2 wt % Tween-20 and 2 wt % acetylcysteine;
(6) the product obtained from step (5) was immersed in 1 L of a defatting solution at 25° C. for 16 h, wherein the defatting solution was a mixed solution of a mixture of sucrose ester and isopropanol with a 1:1 volume ratio and 2 wt % acetylcysteine;
(7) the product obtained from step (6) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(8) the product obtained from step (7) was cleaned with supercritical carbon dioxide to obtain the biological membrane, wherein the cleaning was carried out at a pressure of 25 MPa and a temperature of 40° C. with a CO₂ flow rate of 5 L/min; and
(9) the biological membrane obtained from step (8) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

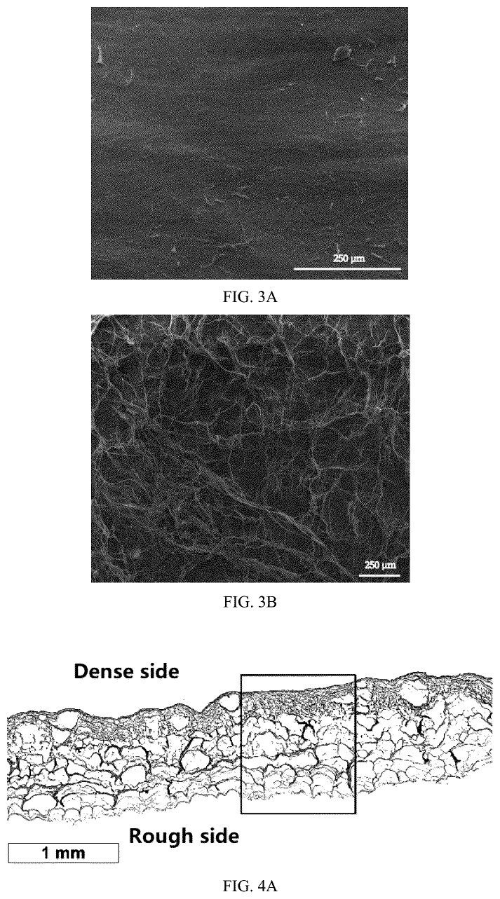

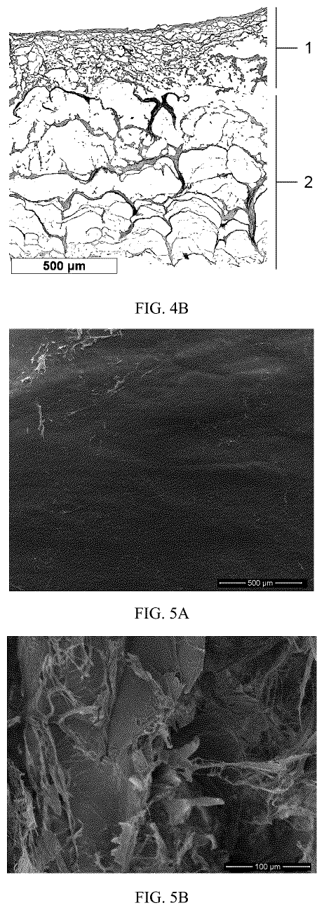

FIG. 3A is an electron microscope image showing the dense side of the biological membrane prepared in Example 1; as shown in FIG. 3A, by using the bladder tissue as the raw material, the dense side of the biological membrane shows a smooth and non-porous condition. FIG. 3B is an electron microscope image showing the rough side of the biological membrane prepared in Example 1; as shown in FIG. 3B, by using the bladder tissue as the raw material, the rough side of the biological membrane shows a loose and porous condition.

### Example 2

This example provides a biological membrane; a preparation method for the biological membrane includes the following steps:
(1) 100 g of fresh porcine stomach tissue was immersed in 1 L of a 5 wt % sodium citrate solution for 4 h;
(2) the product obtained from step (1) was processed at the room temperature by a slicing device to excise a membrane layer of 1 mm thickness from the serosa side of the stomach, wherein specific process parameters of the mechanical cutting device excision were: a voltage of 220 V, a power of 3600 W, a cutting rate of 15 mm/s, and a roughness of tissue surface after excision of less than 100 μm;
(3) 100 g of the product obtained from step (2) was immersed in 1 L of a 1 wt % protease 1398 solution at 25° C. for 5 h;
(4) the product obtained from step (3) was immersed in 1 L of a mixed aqueous solution containing 2 mol/L sodium carbonate and 2% sodium citrate by mass at 10° C. for 4 h;
(5) the product obtained from step (4) was immersed in 1 L of a decellularization solution at 25° C. for 15 h, wherein the decellularization solution was a mixed aqueous solution of 1 wt % sodium dodecylaminopropionate and 2 wt % sodium citrate;
(6) the product obtained from step (5) was immersed in 1 L of a defatting solution at 25° C. for 15 h, wherein the defatting solution was a mixed solution of a mixture of fatty alcohol polyoxyethylene ether and ethylene glycol with a 1:1 volume ratio and 3 wt % sodium citrate;
(7) the product obtained from step (6) was immersed in 1 L of a second enzyme solution at 20-25° C. for 12 h, wherein the second enzyme solution contained: 1% laminarinase, 0.1% DNA enzyme, 0.1% RNA enzyme, 1% protease 1398, and a remainder of water;
(8) the product obtained from step (7) was cleaned with supercritical carbon dioxide to obtain the biological membrane, wherein the cleaning was carried out at a pressure of 30 MPa and a temperature of 35° C. with a CO₂ flow rate of 6 L/min; and
(9) the biological membrane obtained from step (8) was subjected to freeze-drying and irradiation sterilization to obtain the finished product, wherein pre-freezing for the freeze-drying was carried out at a temperature of −40° C. for 1 h, the lyophilizer was set at −20° C. for the freezing temperature, 48 h for the period, and 1 Pa for the vacuum degree, and the irradiation sterilization was Cobalt-60 irradiation sterilization with a irradiation dose of 30 KGy.

FIG. 5A is an electron microscope image showing the dense side of the biological membrane prepared in Example 2; as shown in FIG. 5A, by using the stomach tissue as the raw material, the dense side of the biological membrane shows a smooth and non-porous condition. FIG. 5B is an electron microscope image showing the rough side of the biological membrane prepared in Example 2; as shown in FIG. 5B, by using the stomach tissue as the raw material, the rough side of the biological membrane shows a loose and porous condition.

### Example 3

This example provides a biological membrane; a preparation method for the biological membrane includes the following steps:
(1) 100 g of fresh bovine large intestine tissue was immersed in 1 L of a 15 wt % disodium edetate solution for 2 h;
(2) the product obtained from step (1) was processed at the room temperature by a slicing device to excise a membrane layer of 3 mm thickness from the serosa side of the large intestine tissue, wherein specific process parameters of the mechanical cutting device excision were: a voltage of 220 V, a power of 3600 W, a cutting rate of 15 mm/s, and a roughness of tissue surface after excision of less than 100 μm;
(3) 100 g of the product obtained from step (2) was immersed in 1 L of a 3 wt % trypsin solution at 25° C. for 3 h;
(4) the product obtained from step (3) was immersed in 1 L of a mixed aqueous solution containing 3 mol/L calcium hydroxide and 2% disodium edetate by mass at 15° C. for 1 h;
(5) the product obtained from step (4) was immersed in 1 L of a decellularization solution at 15° C. for 22 h, wherein the decellularization solution was a mixed aqueous solution of 0.5 wt % Triton X-100 and 2 wt % disodium edetate;
(6) the product obtained from step (5) was immersed in 1 L of a defatting solution at 20° C. for 20 h, wherein the defatting solution was a mixed solution of a 4 wt % sodium dodecyl sulfate aqueous solution and 2 wt % disodium edetate;
(7) the product obtained from step (6) was immersed in 1 L of a second enzyme solution at 25° C. for 10-12 h, wherein the second enzyme solution contained: 1% non-starch polysaccharidase, 0.5% DNA enzyme, 0.5% RNA enzyme, 1% neutral protease, and a remainder of water;
(8) the product obtained from step (7) was cleaned with supercritical carbon dioxide to obtain the biological membrane, wherein the cleaning was carried out at a pressure of 20 MPa and a temperature of 40° C. with a CO₂ flow rate of 3 L/min; and
(9) the biological membrane obtained from step (8) was subjected to freeze-drying and irradiation sterilization to obtain the finished product, wherein pre-freezing for the freeze-drying was carried out at a temperature of −80° C. for 2 h, the lyophilizer was set at −10° C. for the freezing temperature, 72 h for the period, and 5 Pa for the vacuum degree, and the irradiation sterilization was Cobalt-60 irradiation sterilization with a irradiation dose of 20 KGy.

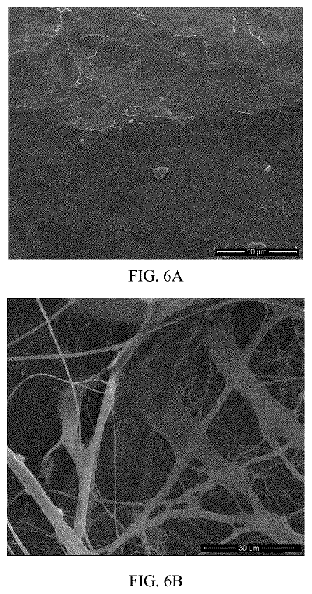

FIG. 6A is an electron microscope image showing the dense side of the biological membrane prepared in Example 3; as shown in FIG. 6A, by using the large intestine tissue as the raw material, the dense side of the biological membrane shows a smooth and non-porous condition. FIG. 6B is an electron microscope image showing the rough side of the biological membrane prepared in Example 3; as shown in FIG. 6B, by using the large intestine tissue as the raw material, the rough side of the biological membrane shows a loose and porous condition.

### Example 4

This example provides a preparation method for a biological membrane, and differs from Example 1 only in that the first enzyme solution in step (3) was a mixed aqueous solution of 2 wt % papain and 3 wt % acetylcysteine;
the second enzyme solution in step (7) was 2% aggrecanase, 0.5% DNA enzyme, 0.5% RNA enzyme, 2% trypsin, 5 wt % acetylcysteine, and a remainder of water.

### Example 5

This example provides a preparation method for a biological membrane, and differs from Example 1 only in that the collagenase inhibitor in step (1) was a 15 wt % tetracycline solution.

### Example 6

This example provides a preparation method for a biological membrane, and differs from Example 1 only in that the alkaline solution in step (4) was a mixed aqueous solution of a 1 mol/L potassium hydroxide solution and 5 wt % penicillamine.

### Example 7

This example provides a preparation method for a biological membrane, and differs from Example 1 only in that acetylcysteine was not added to the alkaline solution in step (4), and the resulting absence was made up with water to 100%.

### Example 8

This example provides a preparation method for a biological membrane, and differs from Example 1 only in that polysaccharidase was not added to the second enzyme solution in step (6), and the resulting absence was made up with water to 100%.

### Example 9

This example provides a preparation method for a biological membrane, and differs from Example 1 only in that the cleaning step with supercritical carbon dioxide in step (8) was carried out at a pressure of 5 Mpa and a temperature of 55° C. with a carbon dioxide flow rate of 15 L/min.

### Example 10

This example provides a preparation method for a biological membrane, and differs from Example 1 only in that the cleaning step with supercritical carbon dioxide in step (8) was carried out at a pressure of 55 Mpa and a temperature of 20° C. with a carbon dioxide flow rate of 0.5 L/min.

### Example 11

This example provides a preparation method for a biological membrane, and differs from Example 1 only in that the animal bladder was processed by a tissue slicing device in step (2) to excise the serosa and subserosa of 2 mm thickness and the muscularis of 1 mm thickness.

### Example 12

This example provides a preparation method for a biological membrane, and differs from Example 1 only in that the animal bladder was processed by a tissue slicing device in step (2) to excise the serosa and subserosa of 1 mm thickness and the muscularis of 3 mm thickness.

### Example 13

This example provides a preparation method for a biological membrane, and differs from Example 2 only in that the porcine stomach was processed by a tissue slicing device in step (2) to excise the serosa and subserosa of 2 mm thickness.

### Example 14

This example provides a preparation method for a biological membrane, and differs from Example 2 only in that the alkaline solution in step (4) was a mixed aqueous solution of 1 mol/L sodium hydroxide and 5% acetylcysteine by mass.

### Example 15

This example provides a preparation method for a biological membrane, and differs from Example 2 only in that the defatting solution in step (6) was: a mixture of fatty alcohol polyoxyethylene ether and ethylene glycol with a volume ratio of 1:1.

### Example 16

This example provides a preparation method for a biological membrane, and differs from Example 3 only in that the animal tissue was selected from animal small intestine.

### Example 17

This example provides a preparation method for a biological membrane, and differs from Example 3 only in that the alkaline solution in step (4) was a mixed aqueous solution of 3 mol/L sodium hydroxide and 2% disodium edetate by mass.

### Example 18

This example provides a preparation method for a biological membrane, and differs from Example 3 only in that the defatting solution in step (6) is a mixed solution of a mixture of sucrose ester and isopropanol with a 1:1 volume ratio and 5 wt % disodium edetate.

### Example 19

This example provides a preparation method for a biological membrane, and differs from Example 3 only in that the second enzyme solution in step (7) contained: a mixed solution of 2% laminarinase, 1% DNA enzyme, 1% RNA enzyme, and 3% trypsin.

### Comparative Example 1

This comparative example provides a biological membrane, and a preparation method for the biological membrane specifically includes the following steps:
(1) 100 g of fresh bladder tissue was cleaned with purified water for later use;
(2) the product obtained from step (1) was processed at the room temperature by a slicing device to excise a membrane layer of 2 mm thickness from the serosa side of the bladder material;
(3) 100 g of the product obtained from step (2) was immersed in 1 L of 2 wt % trypsin solution for 4 h at 25° C.;
(4) the product obtained from step (3) was immersed in 1 L of an aqueous solution containing 1 mol/L sodium hydroxide at 5° C. for 2 h;
(5) the product obtained from step (4) was immersed in 1 L of an aqueous solution of 2 wt % Tween-20 at 25° C. for 16 h;
(6) the product obtained from step (5) was immersed in 1 L of a defatting solution at 25° C. for 16 h, wherein the defatting solution was a mixture of sucrose ester and isopropanol with a volume ratio of 1:1;
(7) the product obtained from step (6) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(8) the product obtained from step (7) was cleaned with supercritical carbon dioxide to obtain the biological membrane, wherein the cleaning was carried out at a pressure of 25 MPa and a temperature of 40° C. with a CO₂ flow rate of 5 L/min; and
(9) the biological membrane obtained from step (8) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 2

This comparative example provides a biological membrane, and a preparation method for the biological membrane specifically includes the following steps:
(1) 100 g of fresh bladder tissue was immersed in 1 L of a 10 wt % acetylcysteine solution for 2 h;
(2) the product obtained from step (1) was processed at the room temperature by a slicing device to excise a membrane layer of 2 mm thickness from the serosa side of the bladder material;
(3) the product obtained from step (2) was immersed in 1 L of an mixed aqueous solution containing 1 mol/L sodium hydroxide and 3% acetylcysteine by mass at 5° C. for 2 h;
(4) 100 g of the product obtained from step (3) was immersed in 1 L of a 2 wt % trypsin solution at 25° C. for 4 h;
(5) the product obtained from step (4) was immersed in 1 L of a decellularization solution at 25° C. for 16 h, wherein the decellularization solution was a mixed aqueous solution of 2 wt % Tween-20 and 2 wt % acetylcysteine;
(6) the product obtained from step (5) was immersed in 1 L of a defatting solution at 25° C. for 16 h, wherein the defatting solution was a mixed solution of a mixture of sucrose ester and isopropanol with a 1:1 volume ratio and 2 wt % acetylcysteine;
(7) the product obtained from step (6) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(8) the product obtained from step (7) was cleaned with supercritical carbon dioxide to obtain the biological membrane, wherein the cleaning was carried out at a pressure of 25 MPa and a temperature of 40° C. with a CO₂ flow rate of 5 L/min; and
(9) the biological membrane obtained from step (8) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 3

This comparative example provides a biological membrane, and a preparation method for the biological membrane specifically includes the following steps:
(1) 100 g of fresh porcine bladder tissue was immersed in 1 L of a 10 wt % acetylcysteine solution for 2 h;
(2) the product obtained from step (1) was processed at the room temperature by a slicing device to excise a membrane layer of 2 mm thickness from the serosa side of the bladder material;
(3) 100 g of the product obtained from step (2) was immersed in 1 L of 2 wt % trypsin solution at 25° C. for 4 h;
(4) the product obtained from step (3) was immersed in 1 L of an alkaline decellularization solution at 15° C. for 6 h, wherein the alkaline decellularization solution was a mixed aqueous solution containing 1 mol/L sodium hydroxide, 2 wt % Tween-20, and 3% acetylcysteine by mass;
(5) the product obtained from step (4) was immersed in 1 L of a defatting solution at 25° C. for 16 h, wherein the defatting solution was a mixed solution of a mixture of sucrose ester and isopropanol with a 1:1 volume ratio and 2 wt % acetylcysteine;
(6) the product obtained from step (5) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(7) the product obtained from step (6) was cleaned with supercritical carbon dioxide to obtain the biological membrane, wherein the cleaning was carried out at a pressure of 25 MPa and a temperature of 40° C. with a CO₂ flow rate of 5 L/min; and
(8) the biological membrane obtained from step (7) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 4

This comparative example provides a biological membrane, and a preparation method for the biological membrane specifically includes the following steps:
(1) 100 g of fresh bladder tissue was immersed in 1 L of a 10 wt % acetylcysteine solution for 2 h;
(2) the product obtained from step (1) was processed at the room temperature by a slicing device to excise a membrane layer of 2 mm thickness from the serosa side of the bladder material;
(3) 100 g of the product obtained from step (2) was immersed in 1 L of 2 wt % trypsin solution for 4 h at 25° C.;
(4) the product obtained from step (3) was immersed in 1 L of a mixed aqueous solution containing 1 mol/L sodium hydroxide and 3% acetylcysteine by mass at 5° C. for 2 h;
(5) the product obtained from step (4) was immersed in 1 L of a decellularization-defatting composite solution at 25° C. for 16 h, wherein the composite solution was a mixed aqueous solution of 2 wt % Tween-20, a mixture of sucrose ester and isopropanol with a 1:1 volume ratio, and 2 wt % acetylcysteine;
(6) the product obtained from step (5) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(7) the product obtained from step (6) was cleaned with supercritical carbon dioxide to obtain the biological membrane, wherein the cleaning was carried out at a pressure of 25 MPa and a temperature of 40° C. with a CO₂ flow rate of 5 L/min; and
(8) the biological membrane obtained from step (7) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 5

This comparative example provides a biological membrane, and a preparation method for the biological membrane specifically includes the following steps:
(1) an animal bladder was processed by a tissue slicing device to remove the serosa, subserosa, and muscularis, and only remained the mucosa and submucosa; the material remained had a total thickness of 1 mm;
(2) the product obtained from step (1) was processed at the room temperature by a slicing device to excise a membrane layer of 2 mm thickness from the serosa side of the bladder material;
(3) 100 g of the product obtained from step (2) was immersed in 1 L of a 2 wt % trypsin solution at 25° C. for 4 h;
(4) the product obtained from step (3) was immersed in 1 L of an mixed aqueous solution containing 1 mol/L sodium hydroxide and 3% acetylcysteine by mass at 5° C. for 2 h;
(5) the product obtained from step (4) was immersed in 1 L of a decellularization solution at 25° C. for 16 h, wherein the decellularization solution was a mixed aqueous solution of 2 wt % Tween-20 and 2 wt % acetylcysteine;
(6) the product obtained from step (5) was immersed in 1 L of a defatting solution at 25° C. for 16 h, wherein the defatting solution was a mixed solution of a mixture of sucrose ester and isopropanol with a 1:1 volume ratio and 2 wt % acetylcysteine;
(7) the product obtained from step (6) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(8) the product obtained from step (7) was cleaned with supercritical carbon dioxide to obtain the biological membrane, wherein the cleaning was carried out at a pressure of 25 MPa and a temperature of 40° C. with a CO₂ flow rate of 5 L/min; and
(9) the biological membrane obtained from step (8) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 6

This comparative example provides a biological membrane, and a preparation method for the biological membrane specifically includes the following steps:
(1) 100 g of fresh bladder tissue was immersed in 1 L of a 10 wt % acetylcysteine solution for 2 h;
(2) the product obtained from step (1) was processed at the room temperature by a slicing device to excise a membrane layer of 2 mm thickness from the serosa side of the bladder material;
(3) the product obtained from step (2) was immersed in 1 L of an mixed aqueous solution containing 1 mol/L sodium hydroxide and 3% acetylcysteine by mass at 5° C. for 2 h;
(4) the product obtained from step (3) was immersed in 1 L of a decellularization solution at 25° C. for 16 h, wherein the decellularization solution was a mixed aqueous solution of 2 wt % Tween-20 and 2 wt % acetylcysteine;
(5) the product obtained from step (4) was immersed in 1 L of a defatting solution at 25° C. for 16 h, wherein the defatting solution was a mixed solution of a mixture of sucrose ester and isopropanol with a 1:1 volume ratio and 2 wt % acetylcysteine;
(6) the product obtained from step (5) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(7) the product obtained from step (6) was cleaned with supercritical carbon dioxide to obtain the biological membrane, wherein the cleaning was carried out at a pressure of 25 MPa and a temperature of 40° C. with a CO₂ flow rate of 5 L/min; and
(8) the biological membrane obtained from step (7) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 7

This comparative example provides a biological membrane, and a preparation method for the biological membrane specifically includes the following steps:
(1) 100 g of fresh bladder tissue was immersed in 1 L of a 10 wt % acetylcysteine solution for 2 h;
(2) the product obtained from step (1) was processed at the room temperature by a slicing device to excise a membrane layer of 2 mm thickness from the serosa side of the bladder material;
(3) 100 g of the product obtained from step (2) was immersed in 1 L of a 2 wt % trypsin solution at 25° C. for 4 h;
(4) the product obtained from step (3) was immersed in 1 L of a mixed aqueous solution containing 1 mol/L sodium hydroxide and 3% acetylcysteine by mass at 5° C. for 2 h;
(5) the product obtained from step (4) was immersed in 1 L of a decellularization solution at 25° C. for 16 h, wherein the decellularization solution was a mixed aqueous solution of 2 wt % Tween-20 and 2 wt % acetylcysteine;
(6) the product obtained from step (5) was immersed in 1 L of a defatting solution at 25° C. for 16 h, wherein the defatting solution was a mixed solution of a mixture of sucrose ester and isopropanol with a 1:1 volume ratio and 2 wt % acetylcysteine;
(7) the product obtained from step (6) was cleaned with supercritical carbon dioxide to obtain the biological membrane, wherein the cleaning was carried out at a pressure of 25 MPa and a temperature of 40° C. with a CO₂ flow rate of 5 L/min; and
(8) the biological membrane obtained from step (7) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 8

This comparative example provides a biological membrane, and a preparation method for the biological membrane specifically includes the following steps:
(1) 100 g of fresh bladder tissue was immersed in 1 L of a 10 wt % acetylcysteine solution for 2 h;
(2) the product obtained from step (1) was processed at the room temperature by a slicing device to excise a membrane layer of 2 mm thickness from the serosa side of the bladder material;
(3) 100 g of the product obtained from step (2) was immersed in 1 L of a 2 wt % trypsin solution at 25° C. for 4 h;
(4) the product obtained from step (3) was immersed in 1 L of a decellularization solution at 25° C. for 16 h, wherein the decellularization solution was a mixed aqueous solution of 2 wt % Tween-20 and 2 wt % acetylcysteine;
(5) the product obtained from step (4) was immersed in 1 L of a defatting solution at 25° C. for 16 h, wherein the defatting solution was a mixed solution of a mixture of sucrose ester and isopropanol with a 1:1 volume ratio and 2 wt % acetylcysteine;
(6) the product obtained from step (5) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(7) the product obtained from step (6) was cleaned with supercritical carbon dioxide to obtain the biological membrane, wherein the cleaning was carried out at a pressure of 25 MPa and a temperature of 40° C. with a CO₂ flow rate of 5 L/min; and
(8) the biological membrane obtained from step (7) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 9

This comparative example provides a biological membrane, and a preparation method for the biological membrane specifically includes the following steps:
(1) 100 g of fresh bladder tissue was immersed in 1 L of a 10 wt % acetylcysteine solution for 2 h;
(2) the product obtained from step (1) was processed at the room temperature by a slicing device to excise a membrane layer of 2 mm thickness from the serosa side of the bladder material;
(3) 100 g of the product obtained from step (2) was immersed in 1 L of a 2 wt % trypsin solution at 25° C. for 4 h;
(4) the product obtained from step (3) was immersed in 1 L of an mixed aqueous solution containing 1 mol/L sodium hydroxide and 3% acetylcysteine by mass at 5° C. for 2 h;
(5) the product obtained from step (4) was immersed in 1 L of a decellularization solution at 25° C. for 16 h, wherein the decellularization solution was a mixed aqueous solution of 2 wt % Tween-20 and 2 wt % acetylcysteine;
(6) the product obtained from step (5) was immersed in 1 L of a defatting solution at 25° C. for 16 h, wherein the defatting solution was a mixed solution of a mixture of sucrose ester and isopropanol with a 1:1 volume ratio and 2 wt % acetylcysteine;
(7) the product obtained from step (6) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(8) the product obtained from step (7) was cleaned with purified water to obtain the biological membrane; and
(9) the biological membrane obtained from step (8) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 10

This comparative example provides a biological membrane, and a preparation method for the biological membrane specifically includes the following steps:
(1) 100 g of fresh bladder tissue was immersed in 1 L of a 10 wt % acetylcysteine solution for 2 h;
(2) the product obtained from step (1) was processed at the room temperature by a slicing device to excise a membrane layer of 2 mm thickness from the serosa side of the bladder material;
(3) 100 g of the product obtained from step (2) was immersed in 1 L of a 2 wt % trypsin solution at 25° C. for 4 h;
(4) the product obtained from step (3) was immersed in 1 L of an mixed aqueous solution containing 1 mol/L sodium hydroxide and 3% acetylcysteine by mass at 5° C. for 2 h;
(5) the product obtained from step (4) was immersed in 1 L of a defatting solution at 25° C. for 16 h, wherein the defatting solution was a mixed solution of a mixture of sucrose ester and isopropanol with a 1:1 volume ratio and 2 wt % acetylcysteine;
(6) the product obtained from step (5) was immersed in 1 L of a decellularization solution at 25° C. for 16 h, wherein the decellularization solution was a mixed aqueous solution of 2 wt % Tween-20 and 2 wt % acetylcysteine;
(7) the product obtained from step (6) was immersed in 1 L of a second enzyme solution at 25° C. for 12 h, wherein the second enzyme solution contained: 2% aggrecanase, 0.5% DNA enzyme, 0.5% RNA enzyme, 2% papain, and a remainder of water;
(8) the product obtained from step (7) was cleaned with supercritical carbon dioxide to obtain the biological membrane, wherein the cleaning was carried out at a pressure of 25 MPa and a temperature of 40° C. with a CO₂ flow rate of 5 L/min; and
(9) the biological membrane obtained from step (8) was subjected to freeze-drying and irradiation sterilization to obtain the finished product, wherein pre-freezing for the freeze-drying was carried out at a temperature of −80° C. for 1 h, the lyophilizer was set at −40° C. for the freezing temperature, 24 h for the period, and 5 Pa for the vacuum degree, and the irradiation sterilization was Cobalt-60 irradiation sterilization with a irradiation dose of 15 KGy.

### Comparative Example 11

This comparative example provides a biological membrane, and a preparation method for the biological membrane specifically includes the following steps:
(1) 100 g of fresh porcine stomach tissue was cleaned with purified water for later use;
(2) the product obtained from step (1) was processed at the room temperature by a slicing device to excise a membrane layer of 1 mm thickness from the serosa side of the stomach material, wherein specific process parameters of the mechanical cutting device excision were: a voltage of 220 V, a power of 3600 W, a cutting rate of 15 mm/s, and a roughness of tissue surface after excision of less than 100 μm;
(3) 100 g of the product obtained from step (2) was immersed in 1 L of a 1 wt % protease 1398 solution at 25° C. for 5 h;
(4) the product obtained from step (3) was immersed in 1 L of a 2 mol/L sodium carbonate solution at 10° C. for 4 h;
(5) the product obtained from step (4) was immersed in 1 L of a decellularization solution at 25° C. for 15 h, wherein the decellularization solution was a 1 wt % sodium dodecyl aminopropionate;
(6) the product obtained from step (5) was immersed in 1 L of a defatting solution at 25° C. for 15 h, wherein the defatting solution was a mixture of fatty alcohol polyoxyethylene ether and ethylene glycol with a volume ratio of 1:1;
(7) the product obtained from step (6) was immersed in 1 L of a second enzyme solution at 20-25° C. for 12 h, wherein the second enzyme solution contained: 1% laminarinase, 0.1% DNA enzyme, 0.1% RNA enzyme, 1% protease 1398, and a remainder of water;
(8) the product obtained from step (7) was cleaned with supercritical carbon dioxide to obtain the biological membrane, wherein the cleaning was carried out at a pressure of 30 MPa and a temperature of 35° C. with a CO₂ flow rate of 6 L/min; and
(9) the biological membrane obtained from step (8) was subjected to freeze-drying and irradiation sterilization to obtain the finished product, wherein pre-freezing for the freeze-drying was carried out at a temperature of −40° C. for 1 h, the lyophilizer was set at −20° C. for the freezing temperature, 48 h for the period, and 1 Pa for the vacuum degree, and the irradiation sterilization was Cobalt-60 irradiation sterilization with a irradiation dose of 30 KGy.

### Comparative Example 12

This comparative example provides a biological membrane, and a preparation method for the biological membrane specifically includes the following steps:
(1) 100 g of fresh porcine stomach tissue was immersed in 1 L of a 5 wt % sodium citrate solution for 4 h;
(2) the product obtained from step (1) was processed at the room temperature by a slicing device to excise a membrane layer of 1 mm thickness from the serosa side of the stomach, wherein specific process parameters of the mechanical cutting device excision were: a voltage of 220 V, a power of 3600 W, a cutting rate of 15 mm/s, and a roughness of tissue surface after excision of less than 100 μm;
(3) the product obtained from step (2) was immersed in 1 L of a mixed aqueous solution of 2 mol/L sodium carbonate and 2% sodium citrate by mass at 10° C. for 4 h;
(4) 100 g of the product obtained from step (3) was immersed in 1 L of a 1 wt % protease 1398 solution at 25° C. for 5 h;
(5) the product obtained from step (4) was immersed in 1 L of a defatting solution at 25° C. for 15 h, wherein the defatting solution was a mixed solution of a mixture of fatty alcohol polyoxyethylene ether and ethylene glycol with a 1:1 volume ratio and 3 wt % sodium citrate;
(6) the product obtained from step (5) was immersed in 1 L of a decellularization solution at 25° C. for 15 h, wherein the decellularization solution was a mixed aqueous solution of 1 wt % sodium dodecyl aminopropionate and 2 wt % sodium citrate;
(7) the product obtained from step (6) was immersed in 1 L of a second enzyme solution at 20-25° C. for 12 h, wherein the second enzyme solution contained: 1% laminarinase, 0.1% DNA enzyme, 0.1% RNA enzyme, 1% protease 1398, and a remainder of water;
(8) the product obtained from step (7) was cleaned with supercritical carbon dioxide to obtain the biological membrane, wherein the cleaning was carried out at a pressure of 30 MPa and a temperature of 35° C. with a CO₂ flow rate of 6 L/min; and
(9) the biological membrane obtained from step (8) was subjected to freeze-drying and irradiation sterilization to obtain the finished product, wherein pre-freezing for the freeze-drying was carried out at a temperature of −40° C. for 1 h, the lyophilizer was set at −20° C. for the freezing temperature, 48 h for the period, and 1 Pa for the vacuum degree, and the irradiation sterilization was Cobalt-60 irradiation sterilization with a irradiation dose of 30 KGy.

### Comparative Example 13

This comparative example provides a biological membrane, and a preparation method for the biological membrane specifically includes the following steps:
(1) 100 g of fresh porcine stomach tissue was immersed in 1 L of a 5 wt % sodium citrate solution for 4 h;
(2) the product obtained from step (1) was processed at the room temperature by a slicing device to excise a membrane layer of 1 mm thickness from the serosa side of the stomach, wherein specific process parameters of the mechanical cutting device excision were: a voltage of 220 V, a power of 3600 W, a cutting rate of 15 mm/s, and a roughness of tissue surface after excision of less than 100 μm;
(3) 100 g of the product obtained from step (2) was immersed in 1 L of a 1 wt % protease 1398 solution at 25° C. for 5 h;
(4) the product obtained from step (3) was immersed in 1 L of a mixed aqueous solution of 2 mol/L sodium carbonate and 2% sodium citrate by mass at 10° C. for 4 h;
(5) the product obtained from step (4) was immersed in 1 L of a decellularization solution at 25° C. for 15 h, wherein the decellularization solution was a mixed aqueous solution of 1 wt % sodium dodecyl aminopropionate and 2 wt % sodium citrate;
(6) the product obtained from step (5) was immersed in 1 L of a defatting solution at 25° C. for 15 h, wherein the defatting solution was a mixed solution of a mixture of fatty alcohol polyoxyethylene ether and ethylene glycol with a volume ratio of 1:1 and 3 wt % sodium citrate;
(7) the product obtained from step (6) was immersed in 1 L of a second enzyme solution at 20-25° C. for 12 h, wherein the second enzyme solution contained: 1% laminarinase, 0.1% DNA enzyme, 0.1% RNA enzyme, 1% protease 1398, and a remainder of water;
(8) the product obtained from step (7) was cleaned with purified water to obtain the biological membrane; and
(9) the biological membrane obtained from step (8) was subjected to freeze-drying and irradiation sterilization to obtain the finished product, wherein pre-freezing for the freeze-drying was carried out at a temperature of −40° C. for 1 h, the lyophilizer was set at −20° C. for the freezing temperature, 48 h for the period, and 1 Pa for the vacuum degree, and the irradiation sterilization was Cobalt-60 irradiation sterilization with a irradiation dose of 30 KGy.

### Comparative Example 14

This comparative example provides a biological membrane, and a preparation method for the biological membrane specifically includes the following steps:
(1) 100 g of fresh bovine large intestine tissue was cleaned with purified water for later use;
(2) the product obtained from step (1) was processed at the room temperature by a slicing device to excise a membrane layer of 3 mm thickness from the serosa side of the large intestine tissue, wherein specific process parameters of the mechanical cutting device excision were: a voltage of 220 V, a power of 3600 W, a cutting rate of 15 mm/s, and a roughness of tissue surface after excision of less than 100 μm;
(3) 100 g of the product obtained from step (2) was immersed in 1 L of 3 wt % trypsin solution at 25° C. for 3 h;
(4) the product obtained from step (3) was immersed in 1 L of an aqueous solution of 3 mol/L calcium hydroxide at 15° C.;
(5) the product obtained from step (4) was immersed in 1 L of a decellularization solution at 15° C. for 22 h, wherein the decellularization solution was an aqueous solution of 0.5 wt % Triton X-100;
(6) the product obtained from step (5) was immersed in 1 L of a defatting solution at 20° C. for 20 h, wherein the defatting solution was an aqueous solution of 4 wt % sodium dodecyl sulfate;
(7) the product obtained from step (6) was immersed in 1 L of a second enzyme solution at 25° C. for 10-12 h, wherein the second enzyme solution contained: 1% non-starch polysaccharidase, 0.5% DNA enzyme, 0.5% RNA enzyme, 1% neutral protease, and a remainder of water;
(8) the product obtained from step (7) was cleaned with supercritical carbon dioxide to obtain the biological membrane, wherein the cleaning was carried out at a pressure of 20 MPa and a temperature of 40° C. with a CO₂ flow rate of 3 L/min; and
(9) the biological membrane obtained from step (8) was subjected to freeze-drying and irradiation sterilization to obtain the finished product, wherein pre-freezing for the freeze-drying was carried out at a temperature of −80° C. for 2 h, the lyophilizer was set at −10° C. for the freezing temperature, 72 h for the period, and 5 Pa for the vacuum degree, and the irradiation sterilization was Cobalt-60 irradiation sterilization with a irradiation dose of 20 KGy.

### Comparative Example 15

This comparative example provides a biological membrane, and a preparation method for the biological membrane specifically includes the following steps:
(1) 100 g of fresh bovine large intestine tissue was immersed in 1 L of a 15% disodium edetate solution for 2 h;
(2) the product obtained from step (1) was processed at the room temperature by a slicing device to excise a membrane layer of 3 mm thickness from the serosa side of the large intestine tissue, wherein specific process parameters of the mechanical cutting device excision were: a voltage of 220 V, a power of 3600 W, a cutting rate of 15 mm/s, and a roughness of tissue surface after excision of less than 100 μm;
(3) 100 g of the product obtained from step (2) was immersed in 1 L of 3 wt % trypsin solution at 25° C. for 3 h;
(4) the product obtained from step (3) was immersed in 1 L of a mixed aqueous solution of 3 mol/L calcium hydroxide and 2% disodium edetate by mass at 15° C. for 1 h;
(5) the product obtained from step (4) was immersed in 1 L of a decellularization-defatting mixed solution at 15° C. for 22 h, wherein the decellularization-defatting mixed solution was a mixed aqueous solution of 0.5 wt % Triton X-100, 4 wt % sodium dodecyl sulfate, and 2 wt % disodium edetate;
(6) the product obtained from step (5) was immersed in 1 L of a second enzyme solution at 25° C. for 10-12 h, wherein the second enzyme solution contained: 1% non-starch polysaccharidase, 0.5% DNA enzyme, 0.5% RNA enzyme, 1% neutral protease, and a remainder of water;
(7) the product obtained from step (6) was cleaned with supercritical carbon dioxide to obtain the biological membrane, wherein the cleaning was carried out at a pressure of 20 MPa and a temperature of 40° C. with a CO₂ flow rate of 3 L/min; and
(8) the biological membrane obtained from step (7) was subjected to freeze-drying and irradiation sterilization to obtain the finished product, wherein pre-freezing for the freeze-drying was carried out at a temperature of −80° C. for 2 h, the lyophilizer was set at −10° C. for the freezing temperature, 72 h for the period, and 5 Pa for the vacuum degree, and the irradiation sterilization was Cobalt-60 irradiation sterilization with a irradiation dose of 20 KGy.

### Comparative Example 16

This comparative example provides a biological membrane, and a preparation method for the biological membrane specifically includes the following steps:
(1) 100 g of fresh bovine large intestine tissue was immersed in 1 L of a 15 wt % disodium edetate solution for 2 h;
(2) the product obtained from step (1) was processed at the room temperature by a slicing device to excise a membrane layer of 3 mm thickness from the serosa side of the large intestine tissue, wherein specific process parameters of the mechanical cutting device excision were: a voltage of 220 V, a power of 3600 W, a cutting rate of 15 mm/s, and a roughness of tissue surface after excision of less than 100 μm;
(3) 100 g of the product obtained from step (2) was immersed in 1 L of 3 wt % trypsin solution at 25° C. for 3 h;
(4) the product obtained from step (3) was immersed in 1 L of a mixed aqueous solution of 3 mol/L calcium hydroxide and 2% disodium edetate by mass at 15° C. for 1 h;
(5) the product obtained from step (4) was immersed in 1 L of a decellularization solution at 15° C. for 22 h, wherein the decellularization solution was a mixed aqueous solution of 0.5 wt % Triton X-100 and 2 wt % disodium edetate;
(6) the product obtained from step (5) was immersed in 1 L of a defatting solution at 20° C. for 20 h, wherein the defatting solution was a mixed solution of 4 wt % sodium dodecyl sulfate and 2 wt % disodium edetate;
(7) the product obtained from step (6) was cleaned with supercritical carbon dioxide to obtain the biological membrane, wherein the cleaning was carried out at a pressure of 20 MPa and a temperature of 40° C. with a CO₂ flow rate of 3 L/min; and
(8) the biological membrane obtained from step (7) was subjected to freeze-drying and irradiation sterilization to obtain the finished product, wherein pre-freezing for the freeze-drying was carried out at a temperature of −80° C. for 2 h, the lyophilizer was set at −10° C. for the freezing temperature, 72 h for the period, and 5 Pa for the vacuum degree, and the irradiation sterilization was Cobalt-60 irradiation sterilization with a irradiation dose of 20 KGy.

### Performance Test

Various performances of the finished products provided in Examples 1-19 and Comparative Examples 1-16 are tested individually, and the specific test methods and standards are shown below:
(1) Nucleic acid content: performing the test according to the method specified in YY/T 0606.25-2014;
(2) Fat content: performing the test according to the method specified in the first method of GB/T 5009.6-2010;
(3) Tensile strength: performing the test according to the method specified for type 2 specimen in GB/T 1040.3-2006;
(4) Standard collagen content: performing the test according to the method of determining hydroxyproline in Appendix B of YY/T 1511-2017;
(5) Suture tearing force: performing the test according to the method of determining suture tearing force in YY/T 1794-2021;
(6) Remnant organic substances and reagents: performing liquid and gas chromatography methods according to the properties of organic substances and reagents; and
(7) Cytotoxicity: performing the test according to the method specified in GB/T16886.5-2017;

The specific test results are shown in Table 1 below.

TABLE 1

| | | | | | | Remnant | |
|---|---|---|---|---|---|---|---|
| | | | | | | Organic | |
| | Nucleic | | Standard | | Suture | Substances | |
| | Acid | Fat | Collagen | Tensile | Tearing | and | Cyto- |
| | Content | Content | Content | Strength | Force | Reagents | toxicity |
| Specimen | (ng/mg) | (%) | (%) | (MPa) | (N) | (ppm) | (Grade) |
| Example 1 | 5.71 | 0.15 | 99.2 | 11.82 | 15.58 | 0.5 | 0 |
| Example 2 | 6.23 | 0.16 | 98.5 | 9.91 | 13.42 | 0.6 | 0 |
| Example 3 | 7.14 | 0.25 | 98.3 | 9.58 | 18.23 | 0.5 | 0 |
| Example 4 | 6.35 | 0.22 | 98.3 | 8.86 | 12.19 | 0.5 | 0 |
| Example 5 | 5.76 | 0.18 | 98.8 | 11.35 | 11.65 | 0.5 | 0 |
| Example 6 | 7.22 | 0.22 | 98.9 | 9.98 | 13.51 | 0.6 | 0 |
| Example 7 | 6.31 | 0.19 | 98.2 | 10.50 | 15.47 | 0.4 | 0 |
| Example 8 | 7.84 | 0.31 | 98.5 | 11.36 | 16.88 | 0.6 | 0 |
| Example 9 | 6.93 | 0.16 | 99.0 | 10.12 | 14.36 | 0.3 | 0 |
| Example 10 | 7.55 | 0.13 | 98.6 | 9.15 | 14.14 | 0.2 | 0 |
| Example 11 | 7.02 | 0.17 | 97.9 | 8.79 | 14.55 | 0.3 | 0 |
| Example 12 | 6.57 | 0.12 | 98.5 | 8.92 | 12.38 | 0.2 | 0 |
| Example 13 | 7.12 | 0.11 | 99.5 | 9.16 | 14.25 | 0.5 | 0 |
| Example 14 | 6.98 | 0.21 | 98.9 | 10.23 | 13.98 | 0.4 | 0 |
| Example 15 | 6.35 | 0.14 | 99.0 | 9.89 | 13.64 | 0.3 | 0 |
| Example 16 | 6.94 | 0.29 | 99.4 | 12.53 | 16.96 | 0.5 | 0 |
| Example 17 | 5.13 | 0.32 | 98.7 | 11.75 | 17.42 | 0.4 | 0 |
| Example 18 | 5.27 | 0.18 | 98.6 | 10.86 | 18.87 | 0.2 | 0 |
| Example 19 | 5.86 | 0.25 | 98.8 | 12.78 | 16.67 | 0.3 | 0 |
| Comparative | 9.69 | 0.56 | 95.5 | 2.56 | 4.12 | 0.9 | 0 |
| Example 1 | | | | | | | |
| Comparative | 10.5 | 1.23 | 90.0 | 4.64 | 3.68 | 1.2 | 1 |
| Example 2 | | | | | | | |
| Comparative | 11.8 | 3.36 | 93.5 | 7.98 | 2.16 | 0.8 | 1 |
| Example 3 | | | | | | | |
| Comparative | 9.98 | 5.68 | 94.85 | 8.02 | 3.96 | 0.9 | 0 |
| Example 4 | | | | | | | |
| Comparative | 7.51 | 1.42 | 95.2 | 0.14 | 0.023 | 0.6 | 0 |
| Example 5 | | | | | | | |
| Comparative | 15.25 | 0.99 | 96.5 | 8.50 | 3.12 | 1.5 | 1 |
| Example 6 | | | | | | | |
| Comparative | 52.85 | 2.52 | 97.5 | 9.50 | 5.56 | 1.8 | 2 |
| Example 7 | | | | | | | |
| Comparative | 11.50 | 6.77 | 98.5 | 8.00 | 4.36 | 1.6 | 2 |
| Example 8 | | | | | | | |
| Comparative | 10.65 | 5.42 | 96.0 | 5.64 | 2.12 | 15.4 | 2 |
| Example 9 | | | | | | | |
| Comparative | 8.65 | 3.26 | 95.5 | 7.65 | 4.32 | 1.1 | 1 |
| Example 10 | | | | | | | |
| Comparative | 10.05 | 0.65 | 87.5 | 0.58 | 2.35 | 0.99 | 0 |
| Example 11 | | | | | | | |
| Comparative | 11.30 | 5.36 | 91.5 | 8.21 | 6.32 | 1.37 | 1 |
| Example 12 | | | | | | | |
| Comparative | 9.88 | 2.31 | 94.5 | 7.56 | 8.15 | 10.80 | 2 |
| Example 13 | | | | | | | |
| Comparative | 11.36 | 2.45 | 85.0 | 0.36 | 2.87 | 1.08 | 0 |
| Example 14 | | | | | | | |
| Comparative | 13.45 | 4.27 | 92.5 | 6.85 | 5.64 | 1.45 | 1 |
| Example 15 | | | | | | | |
| Comparative | 45.30 | 3.68 | 90.3 | 7.02 | 4.57 | 2.03 | 2 |
| Example 16 | | | | | | | |

As can be seen from the test data in Table 1, the biological membrane obtained by the preparation method of the present application has a nucleic acid content of less than or equal to 10 ng/mg, a fat content of less than or equal to 0.5 wt %, a tensile strength of more than or equal to 8 MPa, a standard collagen content of more than or equal to 95 wt %, a suture tearing force of more than or equal to 10 N, remnant organic substances and reagents of less than or equal to 1 ppm, and no cytotoxicity. The test results sufficiently indicate that the natural biological membrane material made by the method provided in the present application retains the structural framework of extracellular matrix of the muscularis in the lumen-containing organs tissue, and additionally multilayered biological membranes of different thicknesses can be prepared by precisely controlling the excision thicknesses of the serosa, the subserosa and/or the muscularis of the lumen-containing organs tissue; the biological membranes, which selectively remove different thicknesses of layers, have different matrix frameworks and thereby can be competently used for multiple indications and guide the regeneration of multiple tissues. In addition, the biological membrane made by the method provided in the present application has apparent dense layer and loose layer, and the loose layer can better guide the growth of tissue cells and play a role in repair and regeneration.

The applicant declares that the present application illustrates the preparation method for a biological membrane, and the product therefrom and the application thereof through the above embodiments, but the present application is not limited to the above embodiments, which means that the present application is not necessarily relied on the above embodiments to be implemented. It should be clear to those skilled in the art that any improvement of the present application, equivalent substitution of each raw material, and addition of auxiliary ingredients for the product of the present application, selection of specific methods, etc., shall fall within the protection scope and disclosure scope of the present application.

1. A preparation method for a biological membrane, comprising:
(1) treating an animal lumen-containing organs tissue with a collagenase inhibitor;
(2) removing a membrane layer which contains fat accounting for more than or equal to 40% of the total fat content of the animal lumen-containing organs tissue;
(3) treating a product obtained from step (2) with a first enzyme solution;
(4) treating a product obtained from step (3) with an alkaline solution;
(5) treating a product obtained from step (4) with a decellularization solution;
(6) treating a product obtained from step (5) with a defatting liquid solution;
(7) treating the product obtained from step (6) with a second enzyme solution; and
(8) cleaning the product obtained from step (7) with supercritical carbon dioxide to obtain the biological membrane.

2. The preparation method for a biological membrane according to claim 1, wherein the animal lumen-containing organs tissue in step (1) comprises serosa, subserosa, muscularis, submucosa, and mucosa sequentially layered.

3. The preparation method for a biological membrane according to claim 1, wherein the animal lumen-containing organs tissue in step (1) is selected from any one of an animal esophageal tissue, an animal stomach tissue, an animal intestine tissue, an animal urethral tissue, or an animal bladder tissue.

4. The preparation method for a biological membrane according to claim 1, wherein the collagenase inhibitor in step (1) comprises any one or a combination of at least two of acetylcysteine, disodium edetate, penicillamine, medroxyprogesterone acetate, sodium citrate, tetracycline, or doxycycline;
the treatment in step (1) specifically is: treating the animal lumen-containing organs tissue by immersing it in an aqueous solution of the collagenase inhibitor;
the aqueous solution of the collagenase inhibitor has a mass concentration of 1-20 wt %;
the animal lumen-containing organs tissue and the aqueous solution of the collagenase inhibitor have a mass ratio of 1:(5-15); and
the immersion is carried out at a temperature of 20-30° C. for a period of 1-6 h.

5. The preparation method for a biological membrane according to claim 1, wherein the removal in step (2) is carried out in a manner of any one of mechanical cutting device excision, scissors trimming, manual peeling, or physical grinding, preferably mechanical cutting device excision;
specific process parameters for the mechanical cutting device excision comprise: a voltage of 160-265 V, a power of 1200-4000 W, a cutting rate of 5-40 mm/s, and a roughness of tissue surface after excision of less than 100 μm;
the removal in step (2) is removing the membrane layer which contains fat accounting for 40-60% of the total fat content of the animal lumen-containing organs tissue, preferably 44-52%;
the membrane layer removed in step (2) comprises serosa and subserosa;
the membrane layer removed in step (2) further comprises a part of muscularis;
the membrane layer removed in step (2) is serosa and subserosa, and the product obtained is muscularis, submucosa, and mucosa; or the membrane layer removed is serosa, subserosa, and a part of muscularis, and the product obtained is a remaining part of muscularis, submucosa, and mucosa; and
the membrane layer removed in step (2) has a total thickness of 1-3 mm.

6. The preparation method for a biological membrane according to claim 1, wherein the first enzyme solution in step (3) comprises a trypsin solution and/or a protease 1398 solution;
the first enzyme solution in step (3) has a concentration of 0.1-5 wt %;
the treatment in step (3) is carried out in a manner of immersion, and the immersion is carried out at a temperature of 20-30° C. for a period of 1-8 h;
the alkaline solution in step (4) comprises any one or a combination of at least two of a sodium hydroxide solution, a potassium hydroxide solution, a sodium carbonate solution, or a calcium hydroxide solution;
the alkaline solution in step (4) has a concentration of 0.1-5 mol/L;
the alkaline solution in step (4) further comprises 1-5 wt % of a collagenase inhibitor;
the collagenase inhibitor comprises any one or a combination of at least two of acetylcysteine, disodium edetate, penicillamine, medroxyprogesterone acetate, sodium citrate, tetracycline, or doxycycline; and
the treatment in step (4) is carried out in a manner of immersion, and the immersion is carried out at a temperature of 4-25° C. for a period of 1-4 h.

7. The preparation method for a biological membrane according to claim 1, wherein the decellularization solution in step (5) comprises an aqueous solution of a surfactant;
the surfactant comprises any one or a combination of at least two of polysorbate, sodium dodecylaminopropionate, polyethylene glycol octylphenyl ether, or alkylphenol polyoxyethylene ether;
the polysorbate comprises any one or a combination of at least two of polysorbate-20, polysorbate-40, polysorbate-60, or polysorbate-80;
the aqueous solution of the surfactant has a concentration of 0.1-5 wt %;
the decellularization solution further comprises 1-5 wt % of a collagenase inhibitor;
the collagenase inhibitor comprises any one or a combination of at least two of acetylcysteine, disodium edetate, penicillamine, medroxyprogesterone acetate, sodium citrate, tetracycline, or doxycycline; and
the treatment in step (5) is carried out in a manner of immersion, and the immersion is carried out at a temperature of 15-25° C. for a period of 5-24 h.

8. The preparation method for a biological membrane according to claim 1, wherein the defatting liquid solution in step (6) comprises an organic solvent and/or an aqueous solution of a detergent;
the organic solvent comprises any one or a combination of at least two of sucrose ester, fatty alcohol polyoxyethylene ether, alkylphenol polyoxyethylene ether, ethanol, ethylene glycol, ethyl acetate, isopropanol, trichloromethane, or acetone, preferably a mixture of ethylene glycol and isopropanol;
the aqueous solution of the detergent has a concentration of 0.1-5 wt %;
the detergent comprises polyethylene glycol octylphenyl ether and/or sodium dodecyl sulfate;
the defatting liquid solution in step (6) further comprises 1-5 wt % of a collagenase inhibitor;
the collagenase inhibitor comprises any one or a combination of at least two of acetylcysteine, disodium edetate, penicillamine, medroxyprogesterone acetate, sodium citrate, tetracycline, or doxycycline; and
the treatment in step (6) is carried out in a manner of immersion, and the immersion is carried out at a temperature of 10-25° C. for a period of 1-24 h.

9. The preparation method for a biological membrane according to claim 1, wherein the second enzyme solution in step (7) comprises a mixed aqueous solution of a solution of polysaccharidase, a solution of nuclease, and a solution of protease;
the polysaccharidase comprises any one or a combination of at least two of non-starch polysaccharidase, laminarinase, or aggrecanase;
the nuclease comprises a DNA enzyme and/or an RNA enzyme;
the protease comprises any one or a combination of at least two of papain, neutral protease, protease 1398, or cathepsin;
the second enzyme solution in step (7) has a concentration of 0.1-10 wt %; and
the treatment in step (7) is carried out in a manner of immersion, and the immersion is carried out at a temperature of 20-30° C. for a period of 1-24 h.

10. The preparation method for a biological membrane according to claim 1, wherein the cleaning with supercritical carbon dioxide in step (8) is carried out at a pressure of 10-50 MPa;
the cleaning with supercritical carbon dioxide in step (8) is carried out at a temperature of 31-50° C.; and
the cleaning with supercritical carbon dioxide in step (8) is carried out at a CO₂ flow rate of 1-10 L/min.

11. The preparation method for a biological membrane according to claim 1, wherein step (9) is further carried out after step (8): drying and sterilizing the biological membrane obtained from step (8);
the drying is freeze-drying; pre-freezing is carried out at a temperature of-80° C. to −20° C. before the freeze-drying; the freeze-drying is carried out at a temperature of -50-0° C., the freeze-drying is carried out for a period of 24-72 h, and the freeze-drying is carried out at a vacuum degree of 1-10 Pa;
the sterilization is irradiation sterilization;
the irradiation sterilization is specifically Cobalt-60 irradiation sterilization; and
the Cobalt-60 irradiation sterilization is carried out at an irradiation dose of 15-30 KGy.

12. A multilayered natural biological membrane, which is prepared by the preparation method according to claim 1.

13. (canceled)

14. A method of preparing a material for guiding tissue regeneration, comprising the multilayered natural biological membrane according to claim 12.
